(19) United States
(12) Patent Application Publication
(10) Pub. No.: US 2013/0172424 A1
(43) Pub. Date: Jul. 4, 2013

(54) METHODS AND COMPOSITIONS FOR TREATING DIABETES

(71) Applicant: Golden Biotechnology Corporation, Jersey City, NJ (US)

(72) Inventors: Sheng-Yung Liu, New Taipei City (TW); Wu-Che Wen, New Taipei City (TW)

(73) Assignee: GOLDEN BIOTECHNOLOGY CORPORATION, Jersey City, NJ (US)

(21) Appl. No.: 13/730,503

(22) Filed: Dec. 28, 2012

Related U.S. Application Data
(60) Provisional application No. 61/582,155, filed on Dec. 30, 2011.

(51) Int. Cl. C07C 49/753

(52) U.S. Cl. CPC C07C 49/753
USPC 514/690

(57) ABSTRACT

The present invention provides methods and compositions for treating diabetes by cyclohexenone compounds.

### CROSS REFERENCE

This application claims the benefit of U.S. provisional application Ser. No. 61/582,155, filed Dec. 30, 2011, which is incorporated by reference in its entirety.

### BACKGROUND OF THE INVENTION

Diabetes mellitus, often simply referred to as diabetes, is a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger).

There are three main types of diabetes. Type 1 diabetes results from the body's failure to produce insulin, and presently requires the person to inject insulin. Type 1 diabetes also referred to as insulin-dependent diabetes mellitus, IDDM for short, and juvenile diabetes. Type 2 diabetes results from insulin resistance, a condition in which cells fail to use insulin properly, sometimes combined with an absolute insulin deficiency. Type 2 diabetes is formerly referred to as non-insulin-dependent diabetes mellitus, and adult-onset diabetes. Gestational diabetes is when pregnant women, who have never had diabetes before, have a high blood glucose level during pregnancy. Gestational diabetes may precede development of type 2 diabetes.

Other forms of diabetes mellitus include congenital diabetes, which is due to genetic defects of insulin secretion, cystic fibrosis-related diabetes, steroid diabetes induced by high doses of glucocorticoids, and several forms of monogenic diabetes.

The major goal in treating diabetes is to minimize any elevation of blood sugar (glucose) without causing abnormally low levels of blood sugar.

### SUMMARY OF THE INVENTION

In one aspect provides herein for treating diabetes comprising administering to a subject a therapeutically effective amount of a cyclohexenone compound having the structure:

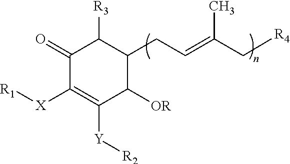

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In another aspect provides herein methods of inhibiting an increase in a blood sugar level in a subject, comprising administering to the subject affected by a disease resulting from hyperglycemia or glucose intolerance or abnormal glucose in need a therapeutically effective amount of a cyclohexenone compound having the structure:

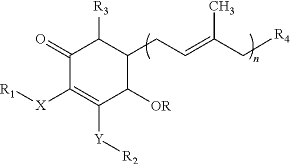

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In another aspect provides herein methods of inhibiting an increase in a blood sugar level in a subject, comprising administering to the subject affected by a disease resulting from hyperglycemia or glucose intolerance or abnormal glucose in need a therapeutically effective amount of a cyclohexenone compound having the structure:

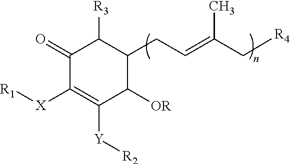

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In another aspect provides herein methods for treating or reducing the risk of a disease resulting from hyperglycemia or glucose intolerance or abnormal glucose of a subject, comprising administering to the subject affected by the disease in need a therapeutically effective amount of a cyclohexenone compound having the structure:

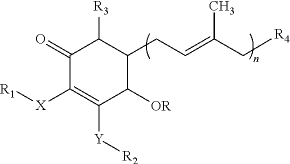

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Diabetes mellitus is a chronic disease which is hard to cure except in very specific situations. Management concentrates on keeping blood sugar levels as close to normal (“euglycemia”) as possible, without causing hypoglycemia. This can usually be accomplished with diet, exercise, and use of appropriate medications (insulin in the case of type 1 diabetes, oral medications as well as possibly insulin in type 2 diabetes).

Type 1 diabetes is typically treated with a combination of regular and NPH insulin, or synthetic insulin analogs. When insulin is used in type 2 diabetes, a long-acting formulation is usually added initially, while continuing oral medications. Doses of insulin are then increased to effect. The invention cyclohexenone compounds, in some embodiments, are obtained from extracts of natural products and provide reduced complications and/or side effects. In some embodiments, provided herein are methods for the treatment of diabetes by administering a cyclohexenone compound provided herein to a subject (e.g. a human). The cyclohexenone compounds provide therapeutic benefit to a subject being treated for diabetes (see Examples 1-7).

In some embodiments, there are provided methods for treating diabetes comprising administering to a subject a therapeutically effective amount of a compound having the structure:

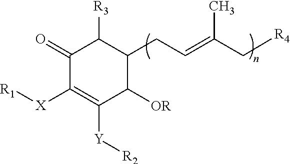

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In some embodiments, the methods inhibit an increase in a blood sugar level in a subject. In some embodiments, the cyclohexenone compound inhibits an increase in a blood sugar level in a subject. In some embodiments, the diabetes is type 1 diabetes, type 2 diabetes or gestational diabetes. In some embodiments, the subject is human. See Examples 2-8.

In some embodiments, the cyclohexenone compound having the structure

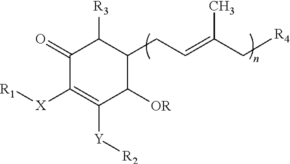

is prepared synthetically or semi-synthetically from any suitable starting material. In other embodiments, the cyclohexenone compound is prepared by fermentation, or the like. For example, Compound 1 (also known as Antroquinonol™ or “Antroq”) or Compound 3, in some instances, is prepared from 4-hydroxy-2,3-dimethoxy-6-methylcyclohexa-2,5-dienone. The non-limited exemplary compounds are illustrated below.

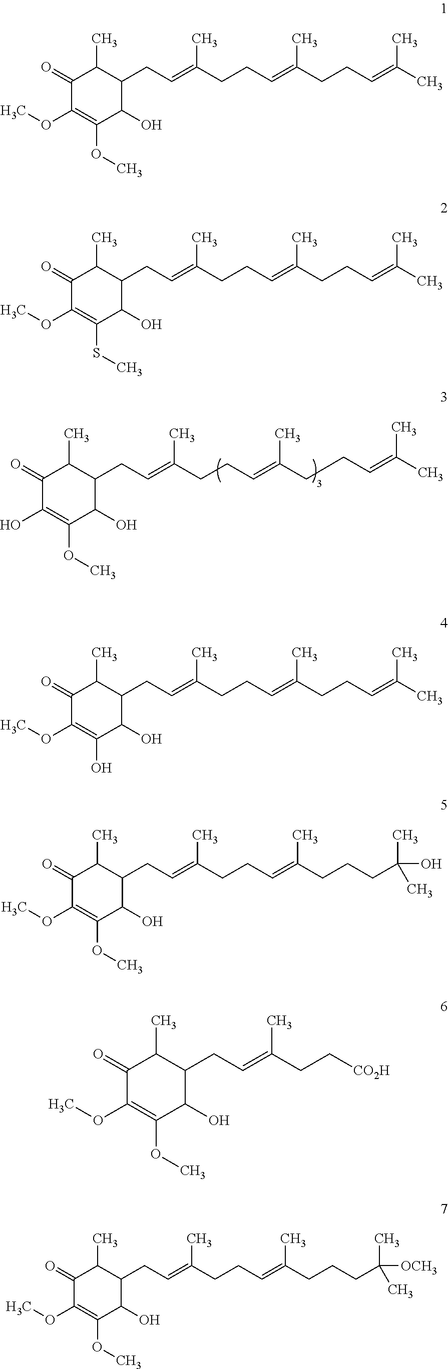

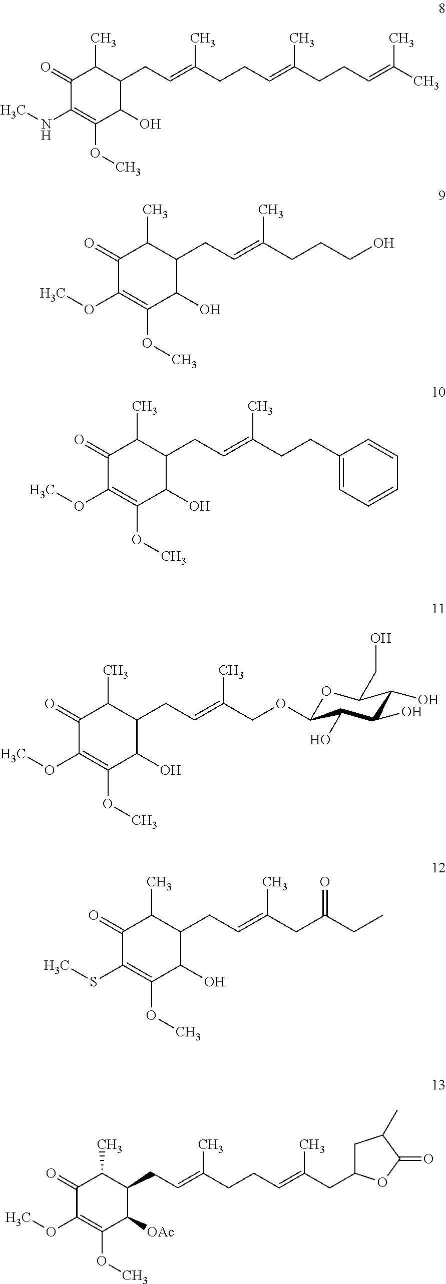

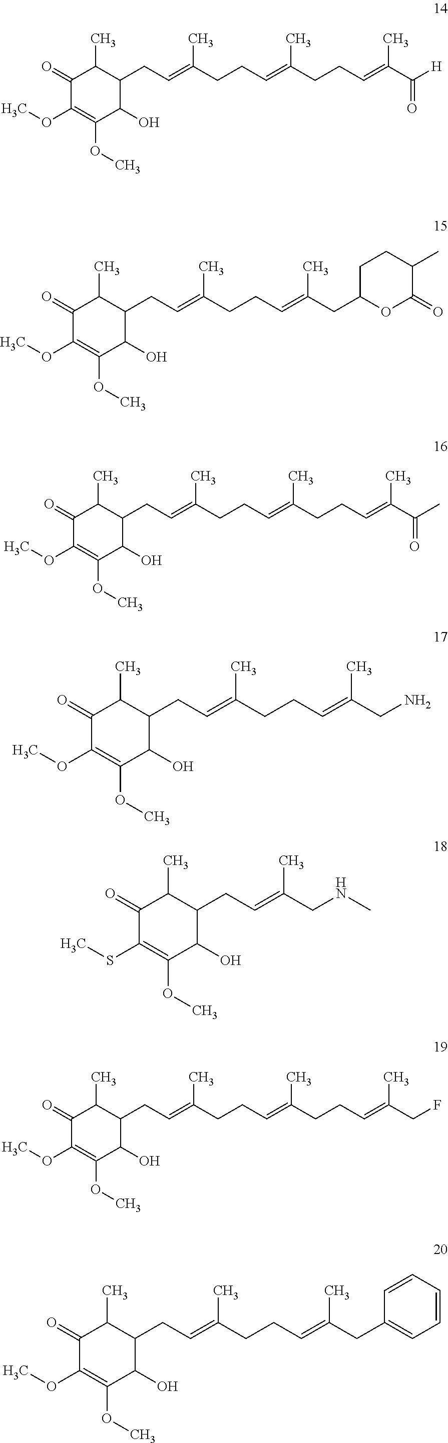

In other embodiments, the cyclohexenone compound having the structure

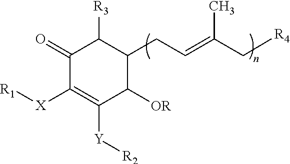

is isolated from the organic solvent extracts of Antrodia camphorata. In some embodiments, the organic solvent is selected from alcohols (e.g., methanol, ethanol, propanol, or the like), esters (e.g., methyl acetate, ethyl acetate, or the like), alkanes (e.g., pentane, hexane, heptane, or the like), halogenated alkanes (e.g., chloromethane, chloroethane, chloroform, methylene chloride, and the like), and the like. For example, exemplary Compounds 1-7 are isolated from organic solvent extracts. In certain embodiments, the organic solvent is alcohol. In certain embodiments, the alcohol is ethanol. In some embodiments, the cyclohexenone compound is isolated from the aqueous extracts of Antrodia camphorata.

In some embodiments, R is a hydrogen, C(═O)C₃H₈, C(═O)C₂H₅, or C(═O)CH₃. In some embodiments, R₁ is a hydrogen or methyl. In certain embodiments, R₂ is a hydrogen, methyl, ethyl, propyl, butyl, pentyl or hexyl. In some embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In some embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₂-C₈alkenyl, C₂-C₈alkynyl, aryl, and glucosyl, wherein 5 or 6-membered lactone, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

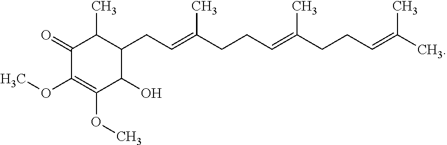 [SMILES: COC1=C(OC)C(O)C(C/C=C(\C)CC/C=C(\C)CCC=C(C)C)C(C)C1=O]

In some embodiments, there are provided methods of inhibiting an increase in a blood sugar level in a subject, comprising administering to the subject affected by a disease resulting from hyperglycemia or glucose intolerance or abnormal glucose in need a therapeutically effective amount of a compound having the structure:

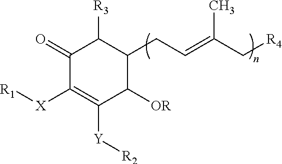

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In some embodiments, the disease of methods of inhibiting an increase in a blood sugar level in a subject resulting from hyperglycemia or glucose intolerance or abnormal glucose is diabetes or a diabetic complication comprising diabetic acidosis, diabetic xanthoma, diabetic amyotrophy, diabetic ketosis, diabetic coma, diabetic gastric disorder, diabetic gangrene, diabetic ulcer, diabetic diarrhea, diabetic microangiopathy, diabetic uterine body sclerosis, diabetic cardiomyopathy, diabetic neuropathy, diabetic nephropathy, diabetic bulla, diabetic cataract, diabetic dermopathy, diabetic scleredema, diabetic retinopathy, necrobiosis lipoidica diabeticorum, or diabetic blood circulation disorder. In some embodiments, wherein the disease resulting from hyperglycemia or glucose intolerance or abnormal glucose is type 1, type 2, or gestational diabetes, or a complication thereof. In certain embodiments, the subject is human.

Impaired glucose tolerance or glucose intolerance is a pre-diabetic state of hyperglycemia that is associated with insulin resistance and increased risk of cardiovascular pathology. IGT may precede type 2 diabetes mellitus by many years. IGT is also a risk factor for mortality.

Hyperglycemia, or high blood sugar is a condition in which an excessive amount of glucose circulates in the blood plasma.

In some embodiments, there are provided methods for treating or reducing the risk of a disease resulting from hyperglycemia or glucose intolerance or abnormal glucose of a subject, comprising administering to the subject affected by the disease in need a therapeutically effective amount of a compound having the structure:

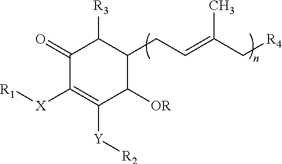

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.
   In some embodiments, the disease resulting from hyperglycemia or glucose intolerance or abnormal glucose is diabetes or a diabetic complication comprising diabetic acidosis, diabetic xanthoma, diabetic amyotrophy, diabetic ketosis, diabetic coma, diabetic gastric disorder, diabetic gangrene, diabetic ulcer, diabetic diarrhea, diabetic microangiopathy, diabetic uterine body sclerosis, diabetic cardiomyopathy, diabetic neuropathy, diabetic nephropathy, diabetic bulla, diabetic cataract, diabetic dermopathy, diabetic scleredema, diabetic retinopathy, necrobiosis lipoidica diabeticorum, or diabetic blood circulation disorder. In certain embodiments, the disease resulting from hyperglycemia or glucose intolerance or abnormal glucose is type 1, type 2, or gestational diabetes, or a complication thereof. In certain embodiments, the cyclohexenone compound inhibits an increase in a blood sugar level in a subject. In some embodiments, the subject is human.

In some embodiments, the cyclohexenone compounds provided herein possess the therapeutic effects of reducing blood sugar in a subject. See Examples 2.

### Certain Pharmaceutical and Medical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group (which means that it does not contain any carbon-carbon double bonds or carbon-carbon triple bonds) or the alkyl group may be an unsaturated alkyl group (which means that it contains at least one carbon-carbon double bonds or carbon-carbon triple bond). The alkyl moiety, whether saturated or unsaturated, may be branched, or straight chain.

The “alkyl” group may have 1 to 12 carbon atoms (whenever it appears herein, a numerical range such as “1 to 12 refers to each integer in the given range; e.g., “1 to 12 carbon atoms” means that the alkyl group may consist of 1 carbon atom, 2 carbon atoms, 3 carbon atoms, etc., up to and including 12 carbon atoms, although the present definition also covers the occurrence of the term “alkyl” where no numerical range is designated). The alkyl group of the compounds described herein may be designated as “C₁-C₈ alkyl” or similar designations. By way of example only, “C₁-C₈ alkyl” indicates that there are one, two, three, four, five, six, seven or eight carbon atoms in the alkyl chain. In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₈ alkyl.

The term “alkylene” refers to a divalent alkyl radical. Any of the above mentioned monovalent alkyl groups may be an alkylene by abstraction of a second hydrogen atom from the alkyl. In one aspect, an alkylene is a C₁-C₁₂alkylene. In another aspect, an alkylene is a C₁-C₈alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

As used herein, the term “aryl” refers to an aromatic ring wherein each of the atoms forming the ring is a carbon atom. Aryl rings are formed by five, six, seven, eight, nine, or more than nine carbon atoms. Aryl groups are optionally substituted. In one aspect, an aryl is a phenyl or a naphthalenyl. In one aspect, an aryl is a phenyl. In one aspect, an aryl is a C₆-C₁₀aryl. Depending on the structure, an aryl group can be a monoradical or a diradical (i.e., an arylene group). In one aspect, an arylene is a C₆-C₁₀ arylene. Exemplary arylenes include, but are not limited to, phenyl-1,2-ene, phenyl-1,3-ene, and phenyl-1,4-ene.

The term “aromatic” refers to a planar ring having a delocalized π-electron system containing 4n+2π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “halo” or, alternatively, “halogen” or “halide” means fluoro, chloro, bromo or iodo.

The term “lactone” refers to a cyclic ester which can be seen as the condensation product of an alcohol group —OH and a carboxylic acid group —COOH in the same molecule. It is characterized by a closed ring consisting of two or more carbon atoms and a single oxygen atom, with a ketone group ═O in one of the carbons adjacent to the other oxygen.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles may be substituted with one or two oxo (═O) moieties, such as pyrrolidin-2-one.

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (i.e., an alkenylene group). In some embodiments, alkenyl groups are optionally substituted. Illustrative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkynyl” as used herein, means a straight, branched chain, or cyclic (in which case, it would also be known as a “cycloalkenyl”) hydrocarbon containing from 2-10 carbons and containing at least one carbon-carbon triple bond formed by the removal of four hydrogens. In some embodiments, depending on the structure, an alkynyl group is a monoradical or a diradical (i.e., an alkynylene group). In some embodiments, alkynyl groups are optionally substituted. Illustrative examples of alkynyl include, but are not limited to, ethynyl, propynyl, butynyl, pentynyl, hexynyl, heptynyl, and the like.

The term “alkoxy” as used herein, means an alkyl group, as defined herein, appended to the parent molecular moiety through an oxygen atom. Illustrative examples of alkoxy include, but are not limited to, methoxy, ethoxy, propoxy, 2-propoxy, butoxy, tert-butoxy, pentyloxy, and hexyloxy.

The term “cycloalkyl” as used herein, means a monocyclic or polycyclic radical that contains only carbon and hydrogen, and includes those that are saturated, partially unsaturated, or fully unsaturated. Cycloalkyl groups include groups having from 3 to 10 ring atoms. Representative examples of cyclic include but are not limited to, the following moieties:

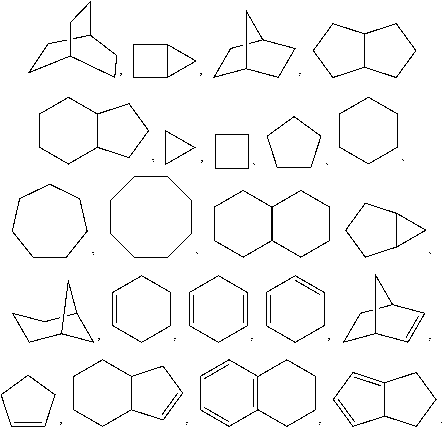

In some embodiments, depending on the structure, a cycloalkyl group is a monoradical or a diradical (e.g., a cycloalkylene group).

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “glucosyl” as used herein, include D- or L-form glucosyl groups, in which the glucosyl group is attached via any hydroxyl group on the glucose ring.

The term “acceptable” with respect to a formulation, composition or ingredient, as used herein, means having no persistent detrimental effect on the general health of the subject being treated.

Antrodia is a genus of fungi in the family Meripilaceae. Antrodia species have fruiting bodies that typically lie flat or spread out on the growing surface, with the hymenium exposed to the outside; the edges may be turned so as to form narrow brackets. Most species are found in temperate and boreal forests, and cause brown rot. Some of the species in this genus are have medicinal properties, and have been used in Taiwan as a traditional medicine.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound (i.e., a cyclohexenone compound described herein) and a co-agent, are both administered to a patient simultaneously in the form of a single entity or dosage. The term “non-fixed combination” means that the active ingredients, e.g. a compound (i.e., a cyclohexenone compound described herein) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound (i.e., a cyclohexenone compound described herein) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, the cyclohexenone compound, or a pharmaceutically acceptable salt, metabolite, solvate or prodrug thereof, is administered parenterally or intravenously. In other embodiments, the cyclohexenone compound, or a pharmaceutically acceptable salt, metabolite, solvate or prodrug thereof, is administered by injection. In some embodiments, the cyclohexenone compound, or a pharmaceutically acceptable salt, metabolite, solvate or prodrug thereof, is administered orally.

### Pharmaceutical Formulation

In some embodiments provide pharmaceutical compositions comprising a therapeutically effective amount of a compound having the structure:

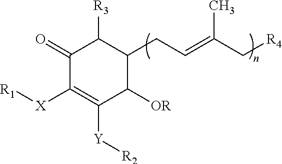

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof; and a pharmaceutically acceptable excipient.

In some embodiments, the cyclohexenone compounds of the pharmaceutical compositions have the structure:

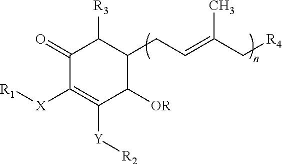

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof.

In some embodiments, R is a hydrogen, C(═O)C₃H₈, C(═O)C₂H₅, or C(═O)CH₃. In some embodiments, each of R₁, R₂ and R₃ independently is a hydrogen, methyl, ethyl, propyl, butyl, pentyl hexyl, heptyl, or octyl. In certain embodiments, R₁ is a hydrogen or methyl. In certain embodiments, R₂ is a hydrogen, methyl, ethyl, propyl, butyl, pentyl or hexyl. In certain embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, C(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

In certain embodiments, the compound is selected from group consisting of

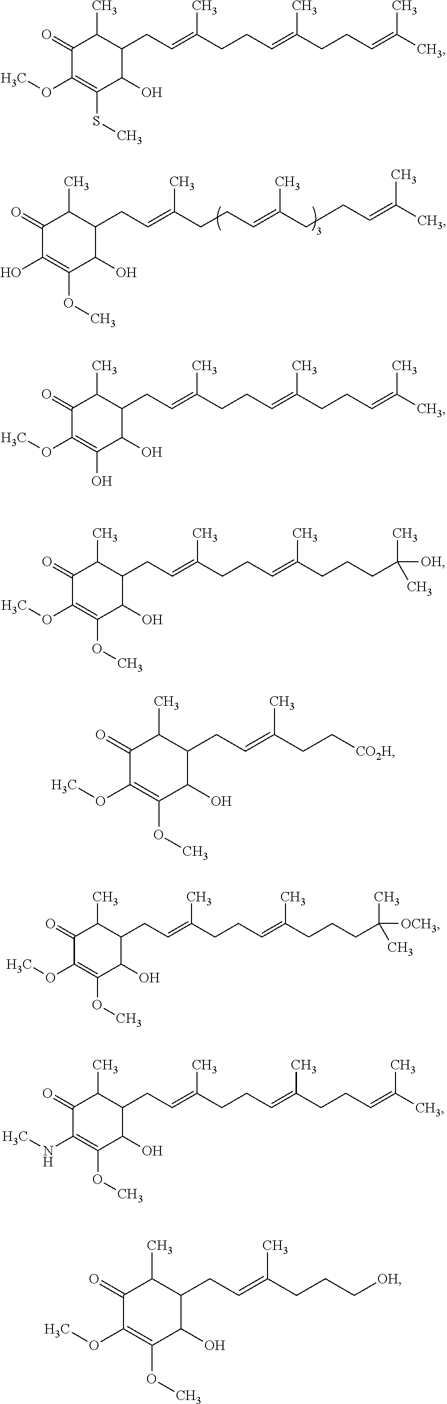

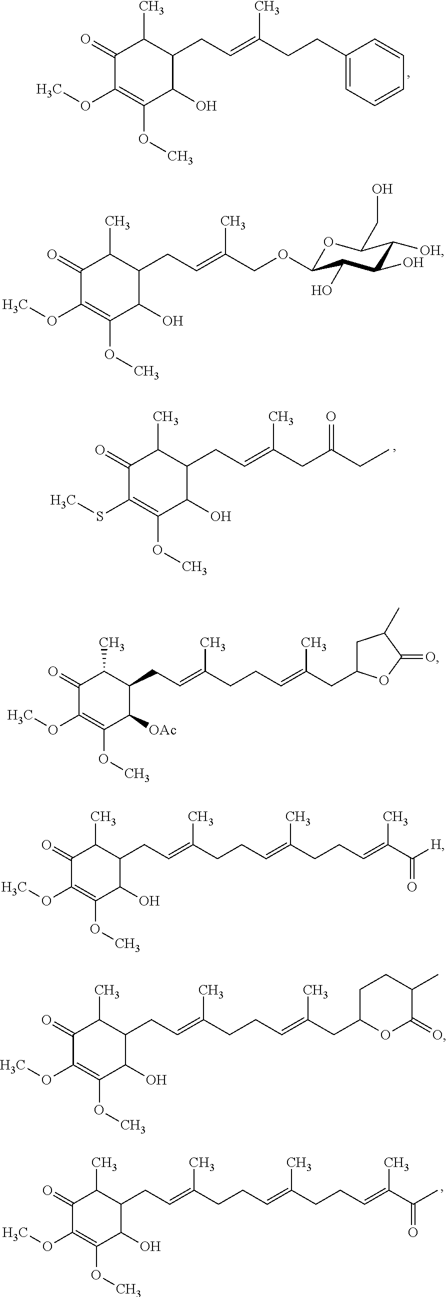

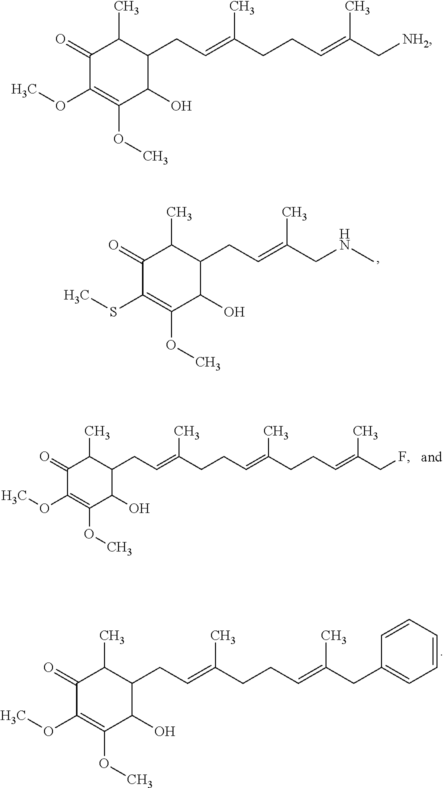

In certain embodiments, the compound is selected from group consisting of

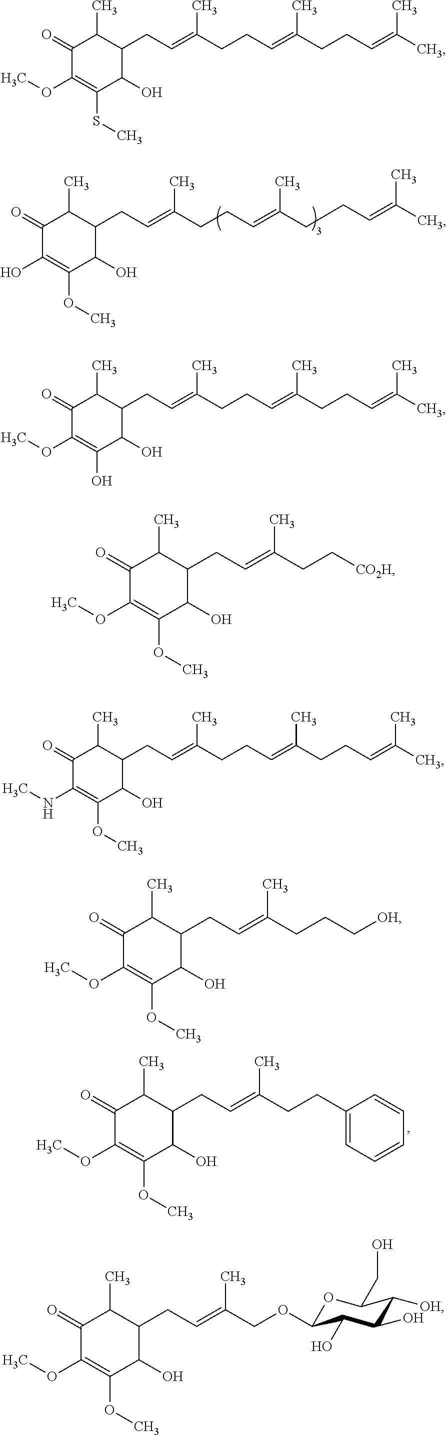

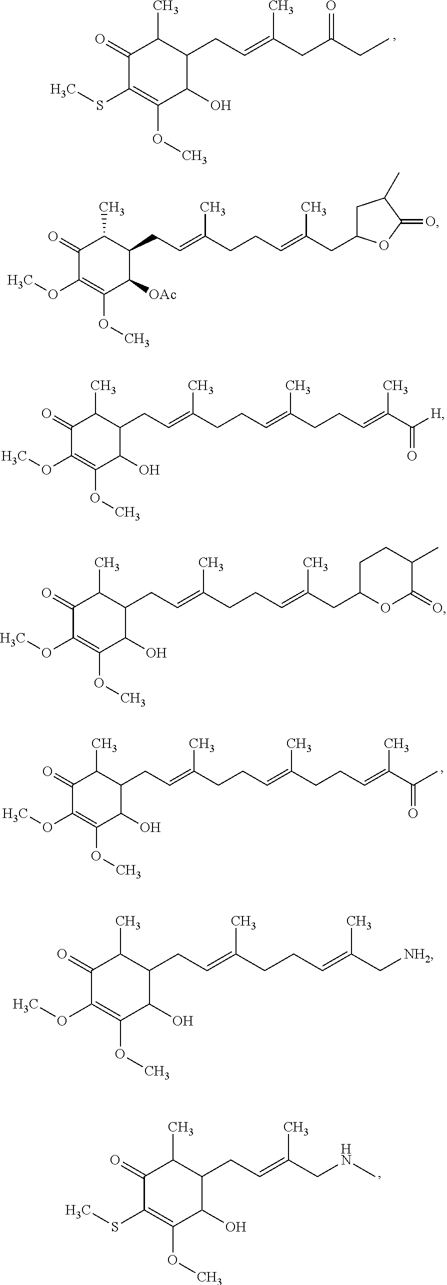

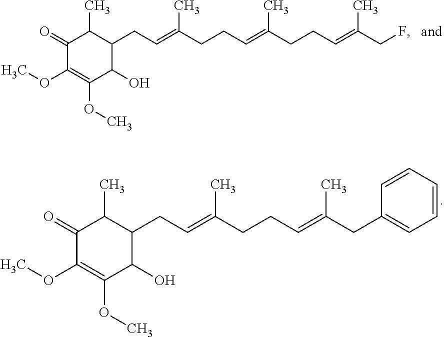 [SMILES: COC1=C(OC)C(O)C(C/C=C(\C)CC/C=C(\C)CC/C=C(\C)CF)C(C)C1=O ; "and" ; COC1=C(OC)C(O)C(C/C=C(\C)CC/C=C(\C)Cc2ccccc2)C(C)C1=O]

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound (i.e., a cyclohexenone compound described herein) and a pharmaceutically acceptable diluent(s), excipient(s), or carrier(s). In certain embodiments, the compounds described are administered as pharmaceutical compositions in which a compound (i.e., a cyclohexenone compound described herein) is mixed with other active ingredients, as in combination therapy. Encompassed herein are all combinations of actives set forth in the combination therapies section below and throughout this disclosure. In specific embodiments, the pharmaceutical compositions include one or more compounds (i.e., a cyclohexenone compound described herein).

A pharmaceutical composition, as used herein, refers to a mixture of a compound (i.e., a cyclohexenone compound described herein) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds (i.e., a cyclohexenone compound described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, a compound (i.e., a cyclohexenone compound described herein) is formulated in an aqueous solution. In specific embodiments, the aqueous solution is selected from, by way of example only, a physiologically compatible buffer, such as Hank's solution, Ringer's solution, or physiological saline buffer. In other embodiments, a compound (i.e., a cyclohexenone compound described herein) is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including a compound (i.e., a cyclohexenone compound described herein), are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of a compound (i.e., a cyclohexenone compound described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, compounds (i.e., cyclohexenone compounds described herein) are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound (i.e., a cyclohexenone compound described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of a compound (i.e., a cyclohexenone compound described herein) per 1 mL of solution. Each injector is capable of delivering only one dose of the compound.

In still other embodiments, the compounds (i.e., cyclohexenone compounds described herein) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds (i.e., cyclohexenone compounds described herein) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of a compound (i.e., a cyclohexenone compound described herein) is accomplished by means of iontophoretic patches and the like. In certain embodiments, transdermal patches provide controlled delivery of a compound (i.e., a cyclohexenone compound described herein). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound (i.e., a cyclohexenone compound described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds (i.e., cyclohexenone compounds described herein) are formulated for administration by inhalation. Various forms suitable for administration by inhalation include, but are not limited to, aerosols, mists or powders. Pharmaceutical compositions of a compound (i.e., a cyclohexenone compound described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include a compound (i.e., a cyclohexenone compound described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, the compounds (i.e., cyclohexenone compounds described herein) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound (i.e., a cyclohexenone compound described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound (i.e., cyclohexenone compounds described herein) described herein as an active ingredient. The active ingredient is in free-acid or free-base form, or in a pharmaceutically acceptable salt form. In addition, the methods and pharmaceutical compositions described herein include the use crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least compound (i.e., cyclohexenone compounds described herein) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound (i.e., cyclohexenone compounds described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### Combination Treatments

In general, the compositions described herein and, in embodiments where combinational therapy is employed, other agents do not have to be administered in the same pharmaceutical composition, and in some embodiments, because of different physical and chemical characteristics, are administered by different routes. In some embodiments, the initial administration is made according to established protocols, and then, based upon the observed effects, the dosage, modes of administration and times of administration is modified by the skilled clinician.

In some embodiments, therapeutically-effective dosages vary when the drugs are used in treatment combinations. Combination treatment further includes periodic treatments that start and stop at various times to assist with the clinical management of the patient. For combination therapies described herein, dosages of the co-administered compounds vary depending on the type of co-drug employed, on the specific drug employed, on the disease, disorder, or condition being treated and so forth.

It is understood that in some embodiments, the dosage regimen to treat, prevent (reduce the risk), or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein.

Combinations of compounds (i.e., the cyclohexenone compound described herein) with other diabetes therapeutic agents are intended to be covered. In some embodiments, examples of diabetes therapeutic agents include, but are not limited to, the following: insulin; sensitizers (e.g., biguanides such as metformin (Glucophage), thiazolidinediones such as pioglitazone (Actos)); secretagogues (e.g., Sulfonylureas such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), gliclazide (Diamicron), and nonsulfonylurea secretagogues such as Meglitinides, repaglinide (Prandin), nateglinide (Starlix)); alpha-glucosidase inhibitors such as miglitol (Glyset) and acarbose (Precose/Glucobay); Injectable Incretin mimetics such as glucagon-like peptide analogs such as Exenatide, Exendin-4, Liraglutide and Taspoglutide; gastric inhibitory peptide analogs such as N-AcGIP, GIP(Lys37)PAL, N-AcGIP(Lys37)PAL, (Pro3)GIP, GLP-1, and the like; other similar peptide analogs such as vildagliptin (Galvus), vildagliptin (Galvus), saxagliptin (Onglyza), and linagliptin (Tradjenta); and Amylin analogues (e.g., pramlintide).

In some embodiments, combinations of compounds (i.e., the cyclohexenone compound described herein) with the following type 1 and/or type 2 diabetes therapeutic agents are used to treat diabetes—NN1250/insulin degludec; Dapagliflozin; Aleglitazar; DiaPep277; GAD-alum/rhGAD65; Otelixizumab; MGA031/hOKT3γ1/teplizumab (Ala-Ala); Arxxant; and the like.

The combinations of the cyclohexenone compounds and other diabetes therapeutic agents described herein encompass additional therapies and treatment regimens with other agents in some embodiments. Such additional therapies and treatment regimens can include another diabetes therapy in some embodiments. Alternatively, in other embodiments, additional therapies and treatment regimens include other agents used to treat adjunct conditions associated with the diabetes or a side effect from such agent in the combination therapy. In further embodiments, adjuvants or enhancers are administered with a combination therapy described herein.

In some embodiments provide compositions for the treatment of diabetes comprising a therapeutically effective amount of a compound having the structure:

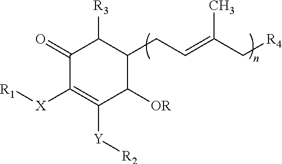

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof; and one or more diabetes therapeutic agents.

### EXAMPLES

### Example 1

### Preparation of the Exemplary Cyclohexenone Compounds

One hundred grams of mycelia, fruiting bodies or mixture of both from Antrodia camphorata were placed into a flask. A proper amount of water and alcohol (70-100% alcohol solution) was added into the flask and were stirred at 20-25° C. for at least 1 hour. The solution was filtered through a filter and 0.45 μm an membrane and the filtrate was collected as the extract.

The filtrate of Antrodia camphorata was subjected to High Performance Liquid chromatography (HPLC) analysis. The separation was performed on a RP18 column, the mobile phase consisted of methanol (A) and 0.3% acetic acid (B), with the gradient conditions of 0-10 min in 95%-20% B, 10-20 min in 20%-10% B, 20-35 min in 10%-10% B, 35-40 min in 10%-95% B, at the flow rate of 1 ml/min. The column effluent was monitored with a UV-visible detector.

The fractions collected at 21.2 to 21.4 min were collected and concentrated to yield compound 5, a product of pale yellow liquid. Compound 5 was analyzed to be 4-hydroxy-5-(11-hydroxy-3,7,11-trimethyldodeca-2,6-dienyl)-2,3-dimethoxy-6-methylcyclohex-2-enone with molecular weight of 408 (Molecular formula: C₂₄H₄₀O₅). ¹H-NMR (CDCl₃) δ (ppm)=1.21, 1.36, 1.67, 1.71, 1.75, 1.94, 2.03, 2.07, 2.22, 2.25, 3.68, 4.05, 5.71 and 5.56. ¹³C-NMR (CDCl₃) δ (ppm): 12.31, 16.1, 16.12, 17.67, 25.67, 26.44, 26.74, 27.00, 30.10, 40.27, 43.34, 59.22, 60.59, 71.8, 120.97, 123.84, 124.30, 131.32, 134.61, 135.92, 138.05, 160.45, and 197.11.

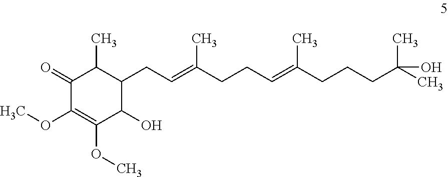 [SMILES: COC1=C(OC)C(O)C(C/C=C(\C)CC/C=C(\C)CCCC(C)(C)O)C(C)C1=O]

### Compound 5: 4-hydroxy-5-(11-hydroxy-3,7,11-trimethyldodeca-2,6-dienyl)-2,3-dimethoxy-6-methylcyclohex-2-enone

The fractions collected at 23.7 to 24.0 min were collected and concentrated to yield compound 7, a product of pale yellow liquid. Compound 7 was analyzed to be 4-hydroxy-2,3-dimethoxy-5-(11-methoxy-3,7,11-trimethyldodeca-2,6-dienyl)-6-methylcyclohex-2-enone with molecular weight of 422 (C₂₅H₄₂O₅). ¹H-NMR (CDCl₃) δ (ppm)=1.21, 1.36, 1.71, 1.75, 1.94, 2.03, 2.07, 2.22, 2.25, 3.24, 3.68, 4.05, 5.12, 5.50, and 5.61. ¹³C-NMR (CDCl₃) δ (ppm): 12.31, 16.1, 16.12, 17.67, 24.44, 26.44, 26.74, 27.00, 37.81, 39.81, 40.27, 43.34, 49.00, 59.22, 60.59, 120.97, 123.84, 124.30, 135.92, 138.05, 160.45 and 197.12.

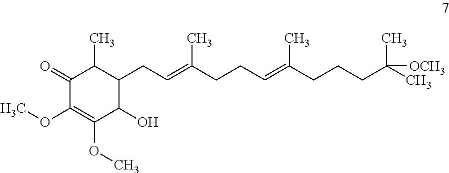 [SMILES: COC1=C(OC)C(O)C(C/C=C(\C)CC/C=C(\C)CCCC(C)(C)OC)C(C)C1=O]

### Compound 7: 4-hydroxy-2,3-dimethoxy-5-(11-methoxy-3,7,11-trimethyldodeca-2,6-dienyl)-6-methylcyclohex-2-enone

The fractions collected at 25 to 30 min were collected and concentrated to yield 4-hydroxy-2,3-dimethoxy-6-methyl-5-(3,7,11-trimethyldodeca-2,6,10-trienyl)cyclohex-2-enone (compound 1), a product of pale yellow brown liquid. The analysis of compound 1 showed the molecular formula of C₂₄H₃₈O₄, molecular weight of 390 with melting point of 48 to 52° C. NMR spectra showed that ¹H-NMR (CDCl₃) δ (ppm)=1.51, 1.67, 1.71, 1.75, 1.94, 2.03, 2.07, 2.22, 2.25, 3.68, 4.05, 5.07, and 5.14; ¹³C-NMR (CDCl₃) δ (ppm)=12.31, 16.1, 16.12, 17.67, 25.67, 26.44, 26.74, 27.00, 39.71, 39.81, 40.27, 43.34, 59.22, 60.59, 120.97, 123.84, 124.30, 131.32, 135.35, 135.92, 138.05, 160.45, and 197.12.

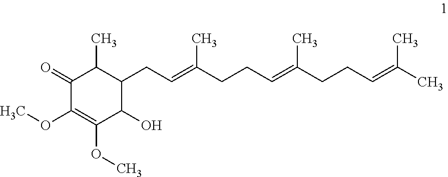 [SMILES: COC1=C(OC)C(O)C(C/C=C(\C)CC/C=C(\C)CCC=C(C)C)C(C)C1=O]

### Compound 1: 4-hydroxy-2,3-dimethoxy-6-methyl-5-(3,7,11-trimethyldodeca-2,6,10-trienyl)cyclohex-2-enone

Compound 6, a metabolite of compound 1, was obtained from urine samples of rats fed with Compound 1 in the animal study. Compound 6 was determined to be 4-hydroxy-2,3-dimethoxy-6-methyl-5-(3-methyl-2-hexenoic acid)cyclohex-2-enone with molecular weight of 312 (C₁₆H₂₄O₆). Compound 4 which was determined as 3,4-dihydroxy-2-methoxy-6-methyl-5-(3,7,11-trimethyldodeca-2,6,10-trienyl)cyclohex-2-enone (molecular weight of 376, C₂₃H₃₆O₄), was obtained when compound 1 was under the condition of above 40° C. for 6 hours.

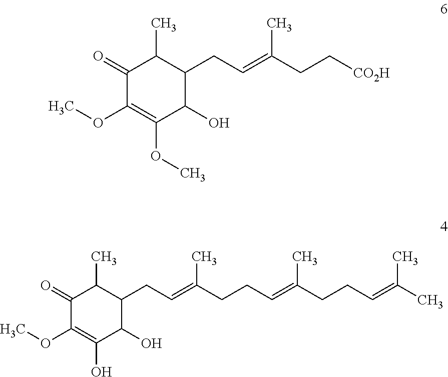 [SMILES: COC1=C(OC)C(O)C(C/C=C(\C)CCC(=O)O)C(C)C1=O ; COC1=C(O)C(O)C(C/C=C(\C)CC/C=C(\C)CCC=C(C)C)C(C)C1=O]

Alternatively, the exemplary compounds may be prepared from 4-hydroxy-2,3-dimethoxy-6-methylcyclohexa-2,5-dienone, or the like. Similarly, other cyclohexenone compounds having the structure

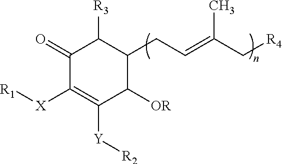

are isolated from Antrodia camphorate or prepared synthetically or semi-synthetically from the suitable starting materials. An ordinary skilled in the art would readily utilize appropriate conditions for such synthesis.

### Example 2

### Treatment of Diabetes Induced by STZ in Rats

The effects of exemplary cyclohexnone compound 1 in treating a subject suffering from diabetes were investigated in a Sprague-Dawley rat model over a four week period. The example describes the results of such experiments.

In vivo models. A rat model of diabetes was established by low-dose streptozotocin (STZ, 60 mg/kg) injection in SD rats. Rats of the same batch fed with normal chow (NRC) were used as a control (n=4).

Study group. Group A: diabetes without treatment group, n=6; Group B: AopE+STZ+Compound 1, n=4. The control group exhibited about 100 mg/dl blood sugar in average before meals. Group A rats exhibited about 400-500 mg/dl blood sugar in average. The rats in Group A also showed typical diabetes symptoms such as high glucose in urine and frequent urination. Rats of Group B (treatment group) have the following blood sugar data before meals:

| ID number | 4^{th} week Blood sugar level | 2^{nd} week blood sugar level |
|---|---|---|
| 1 | 147 mg/dl | 421 mg/dl |
| 2 | 149 mg/dl | 330 mg/dl |
| 3 | 89 mg/dl | 261 mg/dl |
| 4 | 388 mg/dl | 484 mg/dl |

In addition, the rats in Group B showed decreased diabetes symptoms: less glucose in urine, less urine and weight loss.

### Example 3

### Effects of Compound 1 on Blood Glucose Control in Patients with Type 1 Diabetes

The purpose of the study is to evaluate if Compound 1 has an effect on reducing the risk of or treating type 1 diabetes. Especially, if Compound 1 helps to reduce blood glucose; If Compound 1 reduces the occurrence of type 1 diabetes.

Study type: Interventional.

Study design:

Allocation: randomized;

Endpoint Classification Safety/Efficacy Study

Intervention Model: Parallel Assignment

Masking: Double Blind (Subject, Caregiver, Investigator)

### Primary Outcome Measures:

To demonstrate a decrease in post-prandial glucagon release as assessed by glucagon area under the curve (AUC) for 3 hours during 4 hour meal tolerance tests.

Time Frame: Primary outcome measure will be recorded after 16 weeks of treatment. Designated as safety issue: Yes

To demonstrate a decrease in post-prandial glucagon release as assessed by glucagon area under the curve (AUC) for 3 hours during 4 hour meal tolerance tests.

### Secondary Outcome Measures:

Secondary objectives: 1. To demonstrate a 0.3% decrease in A1c in patients with type 1 diabetes after 16 weeks of treatment with Compound 1. 2. To evaluate changes in total, basal and bolus insulin dose while on Compound 1. Time Frame: Secondary outcome measure will be recorded after 16 weeks of treatment. Designated as safety issue: Yes

Criteria in Patients with Diabetes

Ages Eligible for Study: 18 Years to 70 Years (150 subjects)

Genders Eligible for Study: Both

Accepts Healthy Volunteers: No

### Criteria

### Inclusion Criteria:

Signed informed consent before any study-related activities

Male or female aged 18 to 70 years

Type 1 diabetes mellitus duration>1 year

Treatment with MDI or CSII therapy for at least 3 months prior to screening visit; stable insulin dose for the last 1 month

No use of pramlintide, saxagliptin, metformin or sitagliptin for 1 month prior to enrollment

A1c 7.5-10%

Willingness to routinely practice at least 2-4 blood glucose measurements per day

BMI≦35 kg/m2

Ability and willingness to adhere to the protocol including daily oral dose of study drug or placebo and week-long CGM wear

Willing to complete phone and clinic visits

Ability to speak, read and write English

### Exclusion Criteria:

Use of oral, inhaled or pre-mixed insulin

Pregnant or intention to become pregnant during the course of the study not using adequate birth control methods

Severe unexplained hypoglycemia requiring emergency treatment in the previous 3 months

Use of systemic or inhaled corticosteroids

History of hemoglobinopathies

Diagnosis of anemia

Post-renal transplantation, currently undergoing dialysis, creatinine>2.0 mg/dl or a calculated creatinine clearance of <50 mL/min

Advanced retinopathy needing laser procedure or vitrectomy

History of pancreatitis

Extensive skin changes/diseases that inhibit wearing a sensor on normal skin

Known allergy to adhesives

Known allergy to study medication

Participation in another investigational study protocol within 30 days prior to enrollment

Any other condition, as determined by the investigator, which could make the subject unsuitable for the trial, impairs the subject's suitability for the trial, or impairs the validity of the informed consent.

| Arms | Assigned Interventions |
|---|---|
| Compound 1: Experimental | Drug: Compound 1. Dosage form: |
| Intervention: Drug: Compound 1 | 100 mg tablet each. Will take one |
| | capsule a day throughout study |
| Sugar Pill: Placebo Comparator | Drug: Sugar Pill |
| Intervention: Drug: Sugar Pill | 100 mg tablet once a day |

The study provides results of patients who take Compound 1 for type 1 diabetes treatment. These results are clinically significant.

### Example 4

### Efficacy and Safety of Compound 1 in Adult Subjects with Type 2 Diabetes Mellitus

The purpose of this study is to evaluate the safety and effectiveness of multiple doses of Compound 1, once daily (QD), in subjects with type 2 diabetes mellitus.

Study type: Interventional.

Study design:

Allocation: randomized;

Endpoint Classification Safety/Efficacy Study

Intervention Model: Parallel Assignment

Masking: Double Blind (Subject, Caregiver, Investigator, Outcomes Assessor)

### Primary Outcome Measures:

Change from Baseline in Glycosylated Hemoglobin

Time Frame: Week 12 or Final Visit. Designated as safety issue: No

### Secondary Outcome Measures:

Change from baseline in glycosylated hemoglobin. Time Frame: Weeks 4 and 8 or Final Visit; Designated as safety issue: No.

Change from baseline in fasting plasma glucose. Time Frame: Weeks 1, 2, 4, 8 and 12 or Final Visit; Designated as safety issue: No.

Change from baseline in body weight. Time Frame: Weeks 4, 8 and 12 or Final Visit; Designated as safety issue: No.

Number of patients with elevation of alanine aminotransferase greater than three times the Upper Limit of Normal during treatment. Time Frame: Week 12 or Final Visit; Designated as safety issue: No.

Plasma concentrations of Compound via a sparse sampling population approach. Time Frame Week 12 or Final Visit; Designated as safety issue: No.

Criteria in Patients with Diabetes

Ages Eligible for Study: 18 Years to 80 Years (300 subjects)

Genders Eligible for Study: Both

Accepts Healthy Volunteers: No

### Criteria

### Inclusion Criteria:

Historical diagnosis of type 2 diabetes mellitus without the chronic use of antidiabetic therapy and an 8 week history of diet and exercise.

Historical diagnosis of type 2 diabetes mellitus on a stable dose of metformin as mono-therapy for at least 3 months prior to screening.

Glycosylated hemoglobin between 7.5% and 10.0%, inclusive.

Fasting C-peptide concentration is greater than or equal to 0.8 ng per mL.

Any other chronic medications which have been stable for at least 4 weeks prior to Screening.

Body mass index at Screening is greater than or equal to 23 kg/m2 and less than 45 kg/m2.

Able and willing to monitor his or her own blood glucose concentrations with a home glucose monitor.

Females of childbearing potential who are sexually active must agree to use adequate contraception, and can neither be pregnant nor lactating from Screening throughout the duration of the study.

Compliance with single-blinded study medication during the run-in phase is at least 75% and does not exceed 125% based on tablet counts performed by the study staff.

### Exclusion Criteria:

Systolic blood pressure is greater than 160 mm Hg, or diastolic pressure is greater than 100 mm Hg at repeat measurements.

Any history of bladder cancer or has a history of cancer that has been in remission for less than 5 years prior to Screening (a history of basal cell carcinoma or Stage 1 squamous cell carcinoma of the skin is allowed).

Glycosylated hemoglobin is less than 7.5% and greater than 10.0%.

Creatine phosphokinase is greater than or equal to 5 times the upper limit of normal at screening.

Hemoglobin is less than or equal to 12 g per dL for males and less than or equal to 10 g per dL for females.

Alanine aminotransferase and aspartate aminotransferase are greater than or equal to 2.5 upper limit of normal.

Total bilirubin is greater than or equal to 1.5 times the upper limit of normal at screening.

Serum triglyceride concentration is greater than or equal to 400 mg per dL.

Estimated glomerular filtration rate is less than or equal to 60 mL per min using the Modification of Diet in Renal Disease equation or the Cockroft-Gault equation.

Abnormal thyroid-stimulating hormone as defined by central laboratory normals.

Positive test result for hepatitis B surface antigen or hepatitis C antibody.

Urine albumin to creatinine ratio is greater than or equal to 1000 μg per mg at screening.

History of microscopic or macroscopic hematuria.

Two consecutive unexplained positive urinalysis dip-stick and greater than or equal to 3 red blood cells per high-powered field on two consecutive measurements.

History of laser treatment for proliferative diabetic retinopathy within 6 months prior to Screening.

Diabetic gastroparesis that in the investigator's opinion is moderate or severe and hence may impair absorption of study medication.

The subject has New York Heart Association Class III or IV heart failure.

Has had coronary angioplasty, coronary stent placement, coronary bypass surgery, myocardial infarction, unstable angina pectoris, clinically significant abnormal electrocardiogram, cerebrovascular accident or transient ischemic attack within 6 months prior or at Screening.

History of any hemoglobinopathy that may affect determination of glycosylated hemoglobin.

Received treatment with probucol within 1 year of randomization.

Donated or received any blood products within 12 weeks prior to Screening.

Received treatment for greater than 7 days within 8 weeks prior to randomization or is required to take or continues taking any disallowed medication, prescription medication, herbal treatment or over-the counter medication that may interfere with evaluation of the study medication, including:

oral or systemically injected glucocorticoids

Prescription or over the counter weight-loss drugs

Peroxisome proliferator-activated receptor agonists, including fibric acid derivatives

Niacin

Ezetemibe

Bile-acid binding agents

warfarin

phenyloin

any alteration in lipid-lowering medication (change in dosage or drug)

Chronically Treated with Insulin.

Received any investigation drug within 4 weeks prior to Screening.

History of infection with hepatitis B, hepatitis C, or human immunodeficiency virus.

Hypersensitive to Compound 1 or its excipients.

History of drug abuse or a history of alcohol abuse within 2 years prior to Screening.

Any other physical or psychiatric disease or condition that in the judgment of the investigator may affect life expectancy or may make it difficult to successfully manage and follow the subject according to the protocol.

| Arms | Assigned Interventions |
|---|---|
| Compound 1 50 mg QD: | Drug: Compound 1 |
| Experimental | Compound 1 50 mg, tablets, orally, once daily |
| Intervention: Drug: | and pioglitazone placebo-matching tablets, |
| Compound 1 | orally, once daily for up to 12 weeks |
| Compound 1 100 mg | Drug: Compound 1 |
| QD: Experimental | Compound 1 100 mg, tablets, orally, once daily |
| Intervention: Drug: | and pioglitazone placebo-matching tablets, |
| Compound 1 | orally, once daily for up to 12 weeks |
| Compound 1 200 mg | Drug: Compound 1 |
| QD: Experimental | Compound 1 200 mg, tablets, orally, once daily |
| Intervention: Drug: | and pioglitazone placebo-matching tablets, |
| Compound 1 | orally, once daily for up to 12 weeks |
| Pioglitazone 30 mg QD: | Drug: Pioglitazone |
| Active Comparator | Pioglitazone 30 mg, tablets, orally, once daily |
| Intervention: Drug: | and Compound 1 placebo-matching tablets, |
| Pioglitazone | orally, once daily for up to 12 weeks |
| Placebo: Placebo | Drug: Placebo |
| Comparator | Compound 1 placebo-matching tablets, orally, |
| Intervention: Drug: | and pioglitazone placebo-matching tablets, |
| Placebo | orally, once daily for up to 12 weeks |

The study provides results of patients who take Compound 1 for type 2 diabetes treatment. These results are clinically significant.

### Example 5

### Parenteral Formulation

To prepare a parenteral pharmaceutical composition suitable for administration by injection, 100 mg of a compound or its salt described herein is dissolved in DMSO and then mixed with 10 mL of 0.9% sterile saline. The mixture is incorporated into a dosage unit form suitable for administration by injection.

### Example 6

### Oral Formulation

To prepare a pharmaceutical composition for oral delivery, 100 mg of an exemplary Compound 1 was mixed with 100 mg of corn oil. The mixture was incorporated into an oral dosage unit in a capsule, which is suitable for oral administration.

In some instances, 100 mg of a compound described herein is mixed with 750 mg of starch. The mixture is incorporated into an oral dosage unit for, such as a hard gelatin capsule, which is suitable for oral administration.

### Example 7

### Sublingual (Hard Lozenge) Formulation

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 8

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating diabetes comprising administering to a subject a therapeutically effective amount of a compound having the structure: 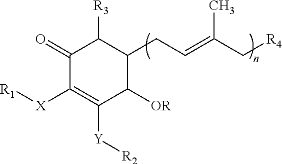
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

2. The method according to claim 1, wherein said method inhibits an increase in a blood sugar level in a subject.

3. The method of claim 2, wherein the diabetes is type 1 diabetes, type 2 diabetes or gestational diabetes.

4. The method according to claim 1, wherein said compound inhibits an increase in a blood sugar level in a subject.

5. A method of inhibiting an increase in a blood sugar level in a subject, comprising administering to the subject affected by a disease resulting from hyperglycemia or glucose intolerance or abnormal glucose in need a therapeutically effective amount of a compound having the structure: 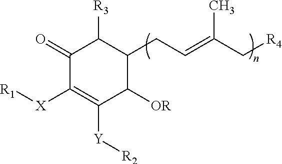
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

6. The method according to claim 5, wherein the disease resulting from hyperglycemia or glucose intolerance or abnormal glucose is diabetes or a diabetic complication comprising diabetic acidosis, diabetic xanthoma, diabetic amyotrophy, diabetic ketosis, diabetic coma, diabetic gastric disorder, diabetic gangrene, diabetic ulcer, diabetic diarrhea, diabetic microangiopathy, diabetic uterine body sclerosis, diabetic cardiomyopathy, diabetic neuropathy, diabetic nephropathy, diabetic bulla, diabetic cataract, diabetic dermopathy, diabetic scleredema, diabetic retinopathy, necrobiosis lipoidica diabeticorum, or diabetic blood circulation disorder.

7. The method according to claim 5, wherein the disease resulting from hyperglycemia or glucose intolerance or abnormal glucose is type 1, type 2, or gestational diabetes, or a complication thereof.

8. A method for treating or reducing the risk of a disease resulting from hyperglycemia or glucose intolerance or abnormal glucose of a subject, comprising administering to the subject affected by said disease in need a therapeutically effective amount of a compound having the structure: 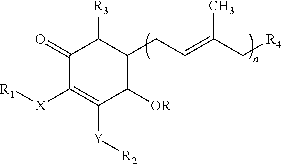
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘCH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

9. The method according to claim 8, wherein the disease resulting from hyperglycemia or glucose intolerance or abnormal glucose is diabetes or a diabetic complication comprising diabetic acidosis, diabetic xanthoma, diabetic amyotrophy, diabetic ketosis, diabetic coma, diabetic gastric disorder, diabetic gangrene, diabetic ulcer, diabetic diarrhea, diabetic microangiopathy, diabetic uterine body sclerosis, diabetic cardiomyopathy, diabetic neuropathy, diabetic nephropathy, diabetic bulla, diabetic cataract, diabetic dermopathy, diabetic scleredema, diabetic retinopathy, necrobiosis lipoidica diabeticorum, or diabetic blood circulation disorder.

10. The method according to claim 8, wherein the disease resulting from hyperglycemia or glucose intolerance or abnormal glucose is type 1, type 2, or gestational diabetes, or a complication thereof.

11. The method of according to claim 8, wherein said compound inhibits an increase in a blood sugar level in a subject.

12. The method of claim 1, wherein said compound, or a pharmaceutically acceptable salt, metabolite, solvate or prodrug thereof, is administered orally, parenterally, intravenously or by injection.

13. The method of claim 1, wherein said subject is human.

14. The method of claim 1, wherein R is a hydrogen, C(═O)C₃H₈, C(═O)C₂H₅, or C(═O)CH₃.

15. The method of claim 1, wherein each of R₁, R₂ and R₃ independently is a hydrogen, methyl, ethyl, propyl, butyl, pentyl, hexyl, heptyl, or octyl.

16. The method of any one of claim 15, wherein R₁ is a hydrogen or methyl.

17. The method of any one of claim 15, wherein R₂ is a hydrogen or methyl.

18. The method of claim 1, wherein R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

19. The method of claim 18, wherein R₄ is CH₂CH═C(CH₃)₂.

20. The method of claim 1, wherein said compound is 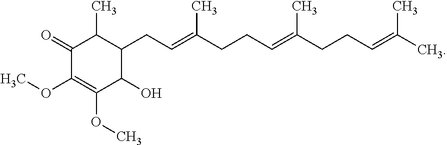 [SMILES: COC1=C(OC)C(O)C(C/C=C(\C)CC/C=C(\C)CCC=C(C)C)C(C)C1=O]
